(19) United States
(12) Patent Application Publication
(10) Pub. No.: US 2004/0143226 A1
(43) Pub. Date: Jul. 22, 2004

(54) Blood collection set with venting mechanism

(75) Inventor: Stewart E. Marsden, Montville, NJ (US)

Correspondence Address: CASELLA & HESPOS LLP, 274 MADISON AVENUE, SUITE 1703, NEW YORK, NY, 10016-0701, US

(73) Assignee: Becton, Dickinson and Company, Franklin Lakes, NJ

(21) Appl. No.: 10/395,304

(22) Filed: Mar. 21, 2003

Related U.S. Application Data
(60) Provisional application No. 60/440,752, filed on Jan. 16, 2003.

(51) Int. Cl. A61M 5/32

(52) U.S. Cl. 604/272

(57) ABSTRACT

The blood collection set includes an IV needle assembly, a length of flexible plastic tubing extending from the IV needle assembly and a non-patient needle assembly. The non-patient needle assembly is formed with a venting plug that permits an outflow of air, while blocking an outflow of blood or other fluids. Thus, the venting mechanism enables air that had existed in interior portions of the blood collection set to be vented, and avoids the need to employ a discard tube.

### RELATED APPLICATIONS

This application claims priority on U.S. Provisional Patent Appl. No. 60/440,752 filed Jan. 16, 2003.

### BACKGROUND OF THE INVENTION

1. Field of the Invention

The subject invention relates to a blood collection set with self-venting features.

2. Description of the Related Art

Phlebotomy procedures often are carried out using a blood collection set. A typical blood collection set includes an IV needle assembly with an IV cannula that has a proximal end, a sharply pointed distal end and a lumen extending between the ends. The needle assembly also includes a plastic IV hub with a proximal end, a distal end, and a passage extending between the ends. The proximal end of the IV cannula is mounted in the passage of the IV hub so that the lumen through the IV cannula communicates with the passage through the IV hub. The needle assembly may further include a shield for shielding the IV cannula after use and a packaging cover for safely covering the IV cannula prior to use. Packaging covers typically are rigid tubes with a proximal end that can be telescoped over the IV cannula and frictionally engaged with the distal end of the IV hub. Shields for blood collection sets have taken many forms. Some shields are telescoped over the IV hub and can be moved from a proximal position where the cannula is exposed to a distal position where the cannula is shielded. Other shields are hinged to the IV hub and can be rotated from an open position where the IV cannula is exposed to a closed position where the IV cannula is shielded. A needle assembly for a blood collection set also may include two flexible wings that project transversely from the IV hub or from the shield. The wings can be folded into face-to-face relationship with one another to effectively define a handle that facilitates manipulation of the needle assembly. The wings then can be rotated away from one another and held against the skin of the patient.

Blood collection sets also include a length of flexible plastic tubing. The tubing has a distal end that is connected to the proximal end of the IV hub. The tubing also has a proximal end that is connected to a plastic fitting. Thus, fluid communication is provided between the lumen of the IV cannula and the plastic fitting at the proximal end of the flexible tubing. The plastic fitting may be a female luer fitting that can be connected to a male luer fitting. The fitting then can be placed in communication with a reservoir or container for collecting a sample of blood.

Phlebotomy procedures often employ evacuated tubes, such as the VACUTAINER® brand of evacuated tubes sold by Becton Dickinson and Company. Evacuated tubes often are used with a tube holder that has a proximal end, a distal end, and a tubular side wall extending between the ends. The proximal end of the holder is widely open and is configured for slidably receiving the evacuated tube. The distal end of the holder typically includes an end wall with a mounting aperture. The mounting aperture includes internal threads or other mounting structures.

The tube holder may be used with a non-patient needle assembly that has a non-patient hub with external surface configurations for mounting in the mounting aperture of the holder. The non-patient needle assembly further includes a non-patient cannula extending proximally from the hub and a multiple sample sleeve telescoped over the non-patient cannula and mounted to the proximal end of the hub. The hub of the non-patient needle assembly can be threaded or otherwise engaged in the mounting aperture of the tube holder so that the non-patient needle and the multiple sample sleeve project into the tube receiving chamber of the holder. In many situations the non-patient needle is pre-mounted in the tube holder.

The blood collection set may be used by mounting the fitting at the proximal end of the flexible plastic tubing to the distal end of the hub of the non-patient needle assembly. The packaging shield that covers the non-patient cannula then may be removed, and the hub of the non-patient needle assembly may be engaged with the tube holder. The medical practitioner then grips the IV needle assembly and removes the packaging cover from the IV cannula. The gripping of the IV needle assembly may include folding the flexible wings into face-to-face engagement and gripping the folded wings between a thumb and forefinger. The pointed distal end of the IV cannula then is urged into a targeted blood vessel. The wings then may be folded into engagement with the skin of the patient and may be taped in position. An evacuated tube then is urged into the open proximal end of the blood collection tube holder so that the non-patient needle pierces the stopper of the evacuated tube. As a result, the blood vessel of the patient is placed in communication with the interior of the evacuated tube, and the pressure differential between the blood vessel and the evacuated tube will generate a flow of blood through the IV cannula, through the passage of the IV hub, through the flexible tubing, through the non-patient hub and finally through the non-patient needle and into the evacuated tube.

It will be appreciated that a significant volume of air must be displaced before blood enters the evacuated tube. This air will be displaced by the flowing blood and will be urged into the evacuated tube. The flow of air into the evacuated tube increases the air pressure in the tube and offsets the pressure differential that generates the flow of blood from the patient to the evacuated tube. Thus, blood flow is slowed. Blood flow into the blood collection tube may stop when the pressure in the tube equals the fluid pressure of the blood. Additionally, the air urged into the blood collection tube can react with the blood or with certain additives in the tube to affect test results that might be performed on the sample in the tube.

Medical practitioners have several approaches for addressing problems relating to air in a blood collection set at the start of a phlebotomy procedure. For example, the first tube of collected blood may be considered a discard tube. Thus, the evacuated tube will remain in communication with the non-patient needle until blood begins to flow into the tube. The tube then will be removed and discarded and a second tube will be inserted into the holder for collecting a sample that can be used reliably. This approach adds to the cost and time of the procedure and wastes blood. Some medical practitioners try to vent air from the system before the first blood collection tube is placed in communication with the non-patient needle. This approach also wastes blood and can lead to contamination or accidental sticks depending upon the method of venting.

The typical needle hub is formed from an opaque plastic material, and plastic tubing often is formed from a translucent plastic material. Neither the opaque plastic material nor the translucent flexible tubing provide a clear indication of venous or arterial access. Blood flow into an evacuated tube does provide an indication of venous or arterial access. However, the initial movement of air into the evacuated tube delays the flow of blood into the evacuated tube. Thus, a medical practitioner may have a delayed indication of venous or arterial access and may incorrectly assume that the blood vessel was not accessed properly. In these situations, the medical practitioner may try to access the blood vessel again even though the initial access was successful. Accordingly, the patient may be subjected to unnecessary trauma during a repeated attempt to access the targeted blood vessel.

### SUMMARY OF THE INVENTION

The invention is a self-venting blood collection set with a self-venting mechanism that permits escape of air while preventing an outflow of fluid, such as blood. Thus, air under venous pressure will be allowed to escape from the blood collection set until blood reaches the venting mechanism. The venting mechanism then will seal to allow blood to be collected into evacuated collection tubes or into other appropriate blood collection receptacles. The venting mechanism may be formed from a hydrophobic material, such as carbon methyl cellulose and preferably is at a location in the blood collection set close to the location that will be placed in communication with the evacuated collection tube or other such container.

The blood collection set preferably includes an IV needle assembly, a length of flexible plastic tubing extending from the IV needle assembly and a non-patient needle assembly. The venting mechanism preferably is disposed on or near the non-patient needle assembly to permit venting of a maximum amount of the air that is in the blood collection set prior to the initiation of a phlebotomy procedure.

The IV needle assembly may comprise an IV hub having a proximal end, a distal end and a passage extending between the ends. The IV needle assembly may further comprise an IV cannula having a proximal end mounted in the passage of the IV hub, a pointed distal end projecting distally from the IV hub and a lumen that communicates with the passage through the IV hub. The flexible tubing may be connected to the proximal end of the IV hub. The IV needle assembly may further include a packaging cover that protectively encloses the IV needle cannula prior to use. The packaging cover is removed immediately prior to use to permit access to the IV cannula. The IV needle assembly may further include a protective shield that is moveable relative to the IV cannula from an open position where the IV cannula is exposed to a closed position where the IV cannula is substantially shielded. The shield protects against accidental sticks with the used IV cannula. A pair of flexible wings may be mounted to the IV hub or to the shield to facilitate manipulation of the IV needle assembly.

The non-patient needle assembly preferably includes a non-patient hub having a proximal end and a distal end. The non-patient needle assembly further includes a non-patient cannula having a distal end securely mounted in the passage through the non-patient hub, a proximal end projecting proximally from the non-patient hub and a lumen that communicates with the passage through the non-patient hub. A multiple sample sleeve may be mounted over the non-patient cannula and secured to the proximal end of the non-patient hub. External portions of the non-patient hub near the proximal end thereof may be formed with an array of external threads or other mounting structure to enable the non-patient needle assembly to be mounted to a collection tube holder or other such medical device. The distal end of the non-patient hub may have a male luer taper that can be placed in communication with a corresponding female luer fitting.

The blood collection set may further include a fitting mounted to the proximal end of the flexible plastic tubing and configured for mating with the distal end of the non-patient hub. For example, the fitting may be a female luer fitting that can be engaged with the male luer taper at the distal end of the non-patient hub. The venting mechanism may extend through the non-patient hub at a location near the distal end of the non-patient needle. The venting mechanism provides communication between the passage through the non-patient hub and the surrounding environment. Alternatively, the venting mechanism may be formed in the fitting mounted to the proximal end of the flexible tubing.

The venting mechanism may comprise a transverse aperture extending through the non-patient plug or through the fitting at the proximal end of the flexible tubing. Additionally, the venting mechanism may comprise a hydrophobic material, such as the above-referenced carbon methyl cellulose mounted in the venting aperture. The hydrophobic material permits air to pass through the hydrophobic material in response to the pressure of blood entering the blood collection set. The air will be urged through the hydrophobic material or other such venting mechanism until the blood reaches the venting mechanism. The blood will not flow through the hydrophobic material and will be at or near to the non-patient needle so that only a minimal amount of air will be collected with the first sample of blood.

The blood collection set of the invention can be used substantially in the conventional manner. In particular, the IV packaging cover is removed from the IV cannula and the medical practitioner accesses a targeted blood vessel with the pointed distal end of the IV cannula. Venous pressure will cause blood to flow through the IV needle assembly and into the flexible plastic tubing. The venous or arterial pressure exceeds the ambient air pressure existing in the flexible plastic tubing and other parts of the blood collection set. Hence, the flowing blood will urge air in the blood collection set out through the venting mechanism on or near the non-patient needle assembly. The hydrophobic material of the venting mechanism and the multiple sample sleeve over the non-patient needle will prevent blood from flowing beyond the blood collection set.

The medical practitioner then may remove the packaging cover from the non-patient needle assembly and may thread or otherwise connect the non-patient hub to the collection tube holder. An evacuated collection tube then can be slid into the open proximal end of the collection tube holder so that the lumen through the non-patient needle is placed in communication with the evacuated interior of the collection tube. The venous or arterial pressure will urge the blood into the collection tube with only a minimal amount of air. As a result, there will be no need for a discard tube or any other blood venting procedure that had been employed in the prior art. Other variations of the above-described blood collection procedure can be employed. For example, the non-patient needle assembly can be connected to the needle holder prior to accessing the blood vessel with the IV needle assembly.

### BRIEF DESCRIPTION OF THE DRAWINGS

FIG. 1 is a perspective view of a blood collection set and collection tube holder in accordance with the subject invention.

FIG. 2 is a top plan view of the blood collection set and collection tube holder shown in FIG. 1.

FIG. 3 is a perspective view of the non-patient needle assembly of the blood collection set.

FIG. 4 is a side elevational view of the non-patient needle assembly, partly in section.

FIG. 5 is a top plan view similar to FIG. 2, but showing an alternate embodiment of the invention.

### DETAILED DESCRIPTION

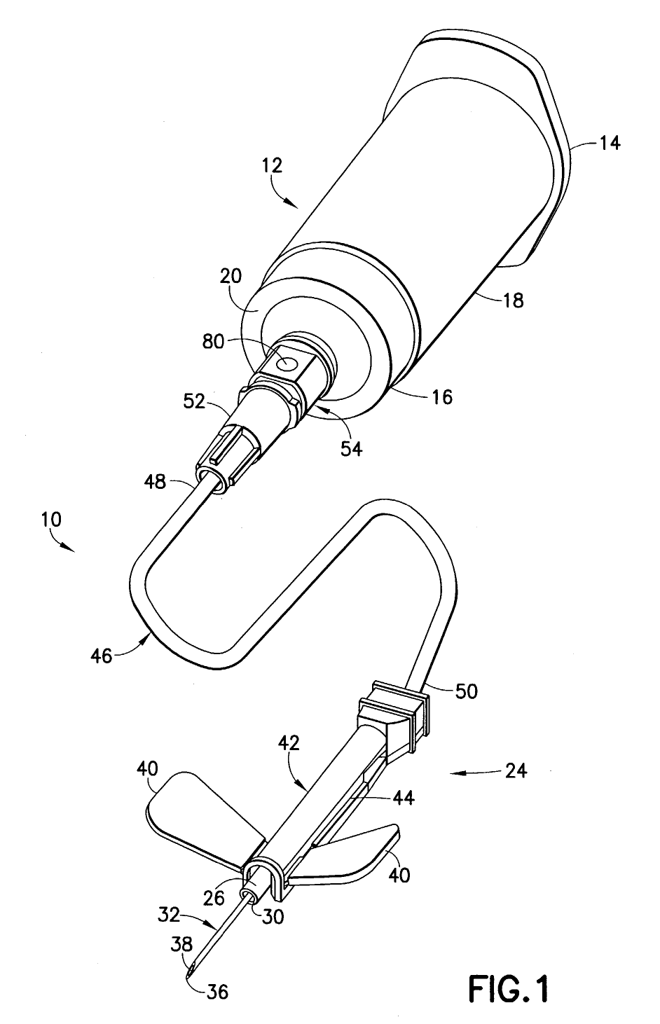

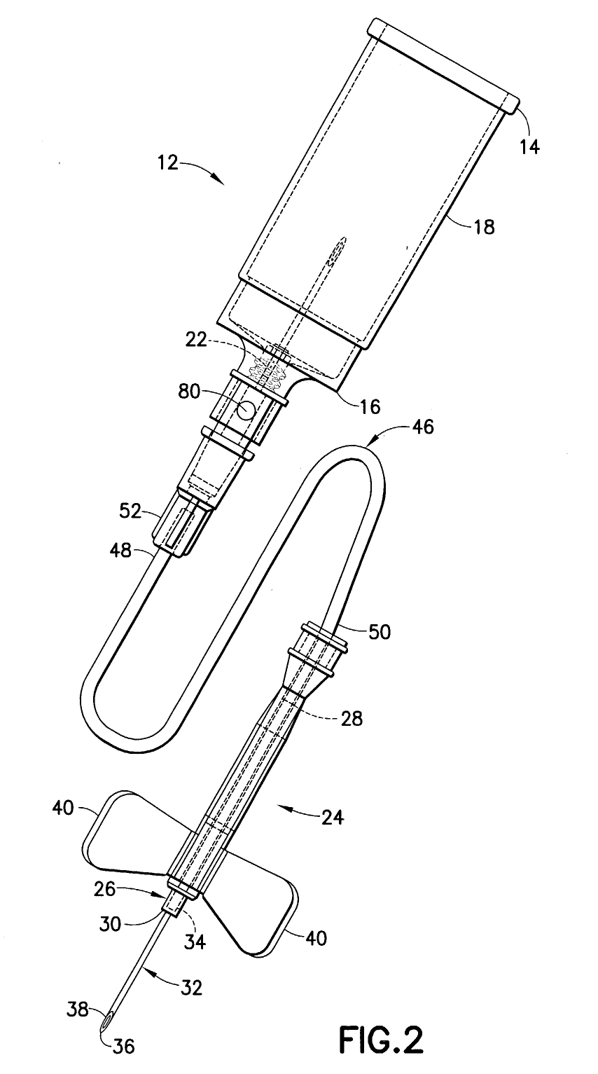

A blood collection set in accordance with the subject invention is identified generally by the numeral 10 in FIGS. 1 and 2. Blood collection set 10 is employed in this embodiment with a collection tube holder 12. Holder 12 has a proximal end 14, a distal end 16 and a tubular sidewall 18 extending between the ends. Proximal end 14 of holder 12 is widely open and defines an entry to a tube receptacle within sidewall 18. Thus, an evacuated collection tube can be slid in a proximal-to-distal direction through open proximal end 14 of holder 12 toward distal end 16. Distal end 16 of holder 12 is characterized by an end wall 20. End wall 20 is formed with an internally threaded mounting aperture 22, as shown in FIG. 2.

Blood collection set 10 includes an IV needle assembly 24 that comprises an IV hub 26. IV hub 26 includes a proximal end 28, a distal end 30 and a passage (not shown) extending between the ends. IV needle assembly 24 further includes an IV cannula 32 with a proximal end 34, a pointed distal end 36 and a lumen 38 extending between the ends. Proximal end 34 of IV cannula 32 is mounted securely in the passage of IV hub 26. Thus, lumen 38 through IV cannula 32 communicates with the passage through IV hub 26. Flexible wings 40 are mounted to IV hub 26 at a location near distal end 30. Wings 40 can be folded into face-to-face relationship with one another for convenient gripping between a thumb and forefinger to enable manipulation of IV needle assembly 24. Wings 40, however, also can be rotated into a substantially coplanar disposition for taping to the skin of a patient.

IV needle assembly 24 further includes a tubular shield 42 that is telescoped over IV hub 26. Shield 42 is formed with transverse slots 44 that slidably receive wings 40. Thus, shield 42 can be slid from a proximal position, as shown in FIGS. 1 and 2 to a distal position. IV cannula 32 is exposed for use when shield 42 is in the proximal position shown in FIGS. 1 and 2. However, IV cannula 32 is substantially surrounded by shield 42 when shield 42 is moved to the distal position. Additionally, slots 44 in shield 42 are configured to lockingly engage wings 40 when shield 42 is in the distal position to prevent or complicate a re-exposure of IV cannula 32. The shield illustrated in FIGS. 1 and 2 is one of many optional shield designs that can be incorporated into blood collection set 10. Other designs may provide wings mounted directly to the shield. Still other designs may provide a hinged shield mounted to IV hub 26. In still other designs, a shield may be entirely separate from IV needle assembly 24 or a shield may not be provided at all.

Blood collection set 10 further includes a length of flexible plastic tubing 46. Tubing 46 includes opposite proximal and distal ends 48 and 50 and a passage extending between the ends. Distal end 50 of tubing 46 is securely mounted to proximal end 28 of IV hub 26 so that the passage through IV hub 26 communicates with the passage through tubing 46. A female luer fitting 52 is securely mounted to proximal end 48 of tubing 46.

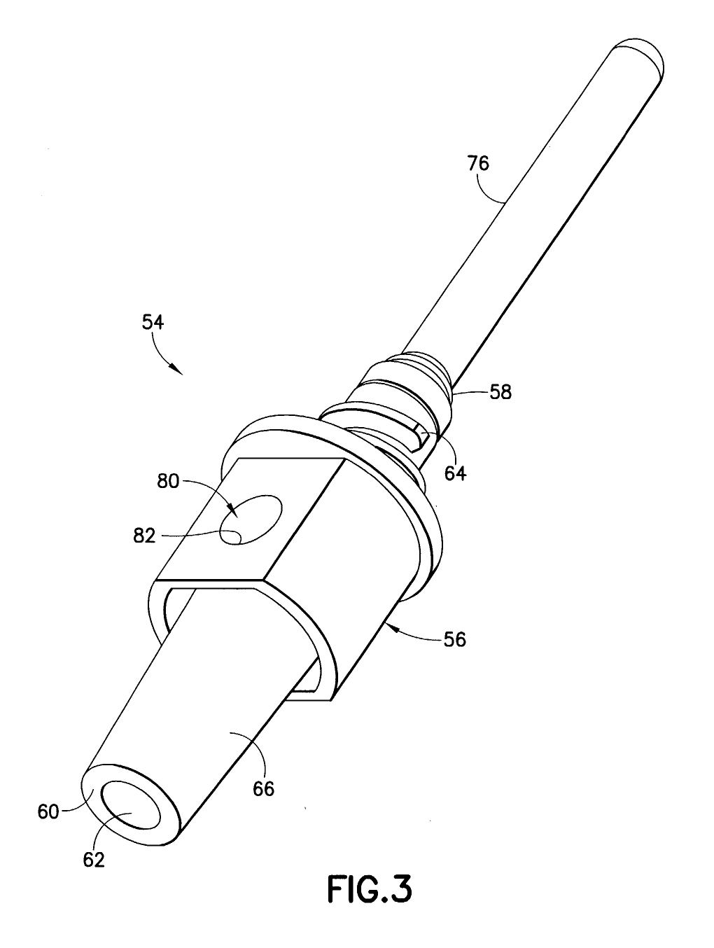

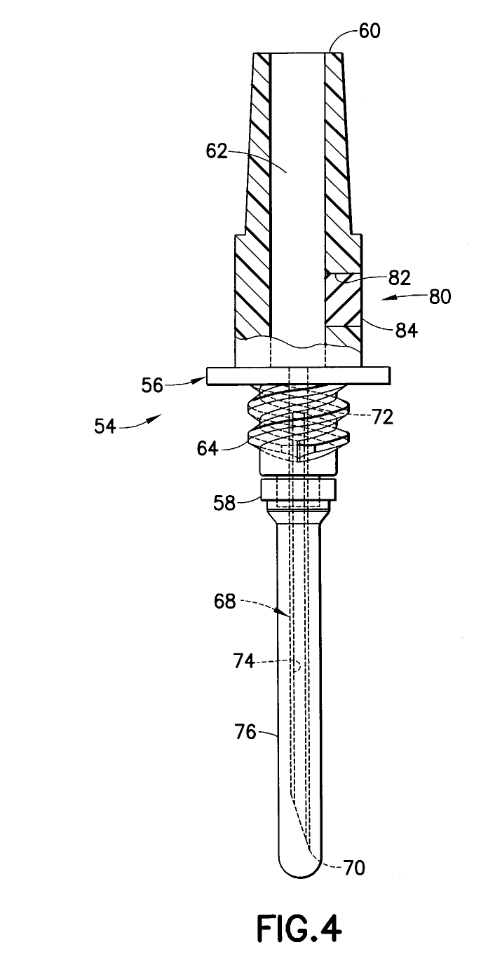

Blood collection set 10 further includes a non-patient needle assembly 54, as shown most clearly in FIGS. 3 and 4. Non-patient needle assembly 54 includes a non-patient hub 56 with a proximal end 58, a distal end 60 and an passage 62 extending between the ends. Exterior surface regions of non-patient hub 56 substantially adjacent proximal end 58 define an array of external threads 64 configured for threaded engagement with the internal threads formed in mounting aperture 22 of collection tube holder 12. External surface regions of non-patient hub 56 adjacent distal end define a male luer taper 66 configured for mating with female luer fitting 52. Non-patient needle assembly 54 further includes a non-patient cannula 68 having a pointed proximal end 70, a distal end 72 and a lumen 74 extending between the ends. Distal end 72 of non-patient cannula 68 is mounted securely in passage 62 through non-patient hub 56 and aligns substantially with external threads 64 on non-patient hub 56. Non-patient needle assembly 54 further includes a multiple sample sleeve 76 mounted over non-patient cannula 68 and securely engaged with proximal end 58 of non-patient hub 56. Multiple sample sleeve 76 effectively functions as a valve that prevents a flow of fluid from non-patient cannula 68. However, multiple sample sleeve 76 can be pierced by pointed proximal end 70 of non-patient cannula 68 in response to forces generated by a stopper on an evacuated collection tube.

Non-patient needle assembly 54 further includes a venting mechanism 80. Venting mechanism 80 includes a transverse aperture 82 extending through non-patient hub 56 at a location between external threads 64 and luer taper 66. Transverse aperture 82 provides communication between passage 62 of non-patient hub 56 and the ambient surroundings. As shown most clearly in FIG. 4, transverse aperture 82 is very close to distal end 72 of non-patient cannula 68. Venting mechanism 80 further includes a venting plug 84 securely mounted in transverse aperture 82. Venting plug 84 is formed from a hydrophobic material, such as carbon methyl cellulose (CMC). Alternatively, venting plug 84 may be formed from high-density polyethylene (HDPE), polytetrafluoroethylene (PTFE), ultra-high molecular weight polyethylene (UHMWPE), Nylon 6, polypropylene (PP), polyvinylidine fluoride (PVDF) or polyethersulfone (PES). Venting plug 84 permits an outflow of air, but prevents an outflow of blood or other fluids.

Blood collection set 10 is employed by folding wings 40 into face-to-face engagement with one another and gripping wings 40 between a thumb and forefinger. Any packaging cover that may be mounted over IV cannula 32 then is removed and discarded. Pointed distal end 36 of IV cannula 32 then is urged into a targeted blood vessel. The healthcare practitioner then may release the grip on wings 40, and if long term access to the blood vessel is required, wings 40 may be taped into face-to-face engagement with the skin of the patient. Blood collection set 10 includes a plurality of internal spaces that will initially be at ambient air pressure. These internal spaces include lumen 38 through IV cannula 32, the passage through IV hub 26, the passage through flexible tubing 46, passage 62 through non-patient hub 56 and lumen 74 through non-patient cannula 68. The venous or arterial access achieved with IV cannula 32 places these interior spaces of blood collection set 10 in communication with the pressure of the blood in the patient. Blood pressure exceeds the ambient air pressure. Accordingly, the pressure of air in the above-referenced internal spaces will increase, and blood will begin to flow into these internal spaces. Prior art systems may reach equilibrium as the air pressure within the blood collection set increases in response to a reduction of volume caused by the inflow of blood. Hence, a portion of the internal spaces in the prior art system may remain filled with air at a pressure substantially equal to the venous or arterial pressure. Stated differently, a prior art system will include its original volume of air in the space between the proximal end of the non-patient needle and the blood that enters the blood collection set. This high-pressure air will escape into the first evacuated collection tube that is placed in communication with the non-patient needle. Hence, the first collection tube employed with prior art systems normally is a discard tube. With the subject invention, however, the communication of blood at venous or arterial pressure with the interior spaces of blood collection set 10 will urge air through venting mechanism 80. Hence, the volume of air in the interior spaces of blood collection set 10 will decrease substantially without a substantial increase in air pressure. Blood will continue to flow into the interior spaces of blood collection set 10, and particularly through the passage of flexible plastic tubing 46 and passage 62 of non-patient hub 56. The outflow of air through venting mechanism 80 will terminate when blood reaches hydrophobic plug 84 of venting mechanism 80. At this point, only a small volume of air will remain in portions of passage 62 between venting mechanism 80 and non-patient cannula 68, as well as the small volumes of air in lumen 74 through non-patient cannula 68 and the volume of air between non-patient cannula 68 and the interior of multiple sample sleeve 76. This volume of air existing proximally of venting mechanism 80 will be very small as compared to the volume of air that had been in flexible plastic tubing 46, but was permitted to escape through venting plug 84.

Use of blood collection set 10 proceeds substantially in a conventional manner by sliding an evacuated collection tube (not shown) into open proximal end 14 of collection tube holder 12. The rubber stopper in the end of the collection tube will displace multiple sample sleeve 76 sufficiently for pointed distal end 70 of non-patient cannula 68 to pierce both multiple sample sleeve 76 and the stopper of the collection tube. A pressure differential then will exist again, and venous or arterial pressure will cause the small volume of air trapped proximally of the blood in non-patient hub 56 into the evacuated tube. Blood then will flow under venous or arterial pressure into the evacuated tube. The small volume of air that initially enters the evacuated tube generally will not require the initial tube to be discarded, and hence blood collection set 10 achieves lower cost and higher efficiency.

After the last sample of blood has been collected, IV cannula 32 is withdrawn from the patient and shield 42 is moved distally to shield IV cannula 32. Blood collection set 10 then may be discarded in an appropriate sharps receptacle.

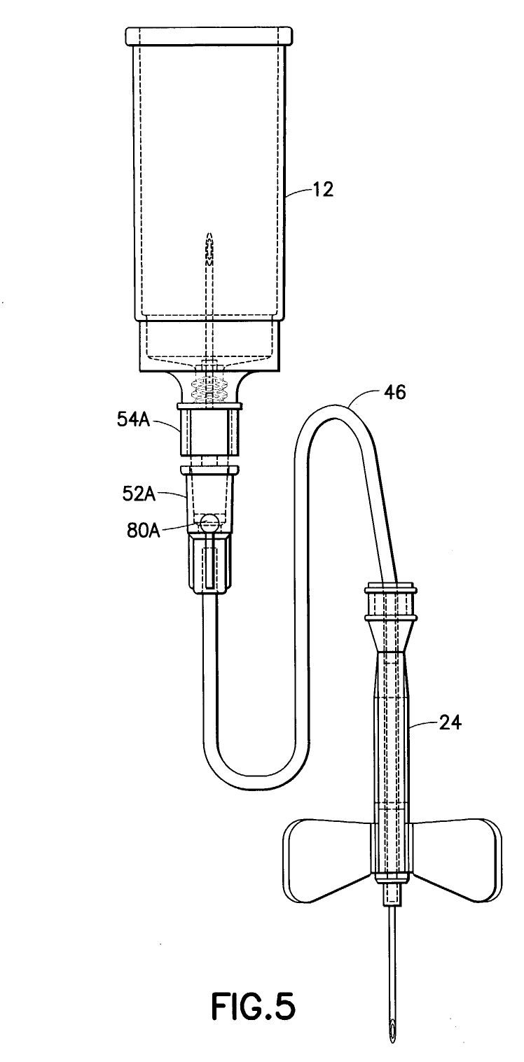

FIGS. 1-4 show an embodiment where venting mechanism 80 is formed in non-patient hub 56 of non-patient needle assembly 54. The embodiment of FIGS. 1-4 provides venting plug 80 virtually as close as possible to the proximal end of blood collection set 10. Similar effects can be achieved by providing venting mechanism 84A in female luer fitting 52A and a non-patient needle assembly 54A with no vent, as shown in FIG. 5. The FIG. 5 embodiment is slightly less efficient than the embodiment of FIGS. 1-4 in that venting mechanism 84A is spaced further from the proximal end of the blood collection set. However the FIG. 5 embodiment enables the use of prior art tube holders and prior art non-patient needle with much better performance than would be achieved with prior art blood collection sets.

What is claimed is:

1. A medical device comprising a first needle assembly having a first hub and a first cannula mounted to said first hub, said first cannula having a lumen extending therethrough, a length of flexible tubing having opposite first and second ends and a passage extending between said ends, said first end of said flexible tubing being mounted to said first hub for providing communication between said lumen of said first cannula and said passage through said flexible tubing, a second hub having a passage extending therethrough and being in fluid communication with said second end of said flexible tubing, a venting mechanism extending through said second hub for providing communication between said passage through said second hub and ambient surroundings, said venting mechanism permitting an outflow of air from said medical device while preventing an outflow of fluid therefrom.

2. The medical device of claim 1, wherein said venting mechanism includes a hydrophobic material.

3. The medical device of claim 2, wherein said hydrophobic material is carbon methyl cellulose.

4. The medical device of claim 1, wherein said second hub further includes structure for placing said passage through said second hub in communication with a collection container.

5. The medical device of claim 4, wherein said structure for placing said passage through said second hub in communication with a collection container comprises a non-patient cannula having opposite proximal and distal ends and a lumen extending between said ends, said distal end of said non-patient cannula being securely mounted to said second hub such that said lumen through said non-patient cannula communicates with said passage through said second hub.

6. The medical device of claim 5, wherein said distal end of said non-patient cannula is in proximity to said venting mechanism.

7. The medical device of claim 5, further comprising a multiple sample sleeve covering said non-patient cannula and securely mounted to said second hub.

8. The medical device of claim 1, wherein said second hub includes a male luer taper formed thereon.

9. The medical device of claim 8, further comprising a female luer fitting securely mounted to said second end of said flexible tubing and being mated with said male luer taper of said second hub.

10. The medical device of claim 1, further comprising a shield for selectively covering said first cannula.

11. A fluid collection set comprising an IV cannula having a lumen extending therethrough, flexible tubing having a passage therethrough communicating with said lumen through said IV cannula, and a fitting having opposite proximal and distal ends and a passage extending between said ends, said distal end of said fitting being configured for placing said passage through said fitting in communication with said passage through said tubing, said proximal end of said fitting being configured for connection with a fluid collection container, a vent extending through said fitting transversely of said passage for providing communication between said passage and ambient surroundings, said vent including a venting material that permits passage of air and that is substantially impervious when exposed to fluid.

12. The blood collection set of claim 11, wherein the venting material is a hydrophobic filter.

13. The blood collection set of claim 11, wherein the venting material is carbon methyl cellulose.

14. The venting mechanism of claim 11, wherein the venting material is selected from the group consisting of high-density polyethylene (HDPE), polytetrafluoroethylene (PTFE), ultra-high molecular weight polyethylene (UHMWPE), Nylon 6, polypropylene (PP), polyvinylidine fluoride (PVDF) and polyethersulfone (PES).

15. The blood collection set of claim 11, wherein the fitting is permanently connected to the tubing.

16. The blood collection set of claim 11, wherein the fitting is releasably placed in communication with said tubing.

17. The blood collection set of claim 16, wherein said distal end defines a male taper, and wherein said blood collection set further includes a female luer connector substantially permanently connected to said tubing, said male luer taper of said fitting being releasably connectable to said female luer connector.

18. The blood collection set of claim 17, wherein the proximal end of said fitting is securely connected to a non-patient cannula, said non-patient cannula having a proximal end externally of said fitting and a distal end secured in said passage through said fitting, said vent being disposed distally of and substantially adjacent to said non-patient cannula.
